(19) United States
(12) Patent Application Publication
(10) Pub. No.: US 2011/0111433 A1
(43) Pub. Date: May 12, 2011

(54) PIEZOELECTRICAL CHARACTERIZATION OF MATERIALS

(75) Inventors: Gil Pogozelich, Katzir (IL); Yaara Kapp-Barnea, Nirit (IL)

(73) Assignee: AMENDIS LTD., Ramat-Gan (IL)

(21) Appl. No.: 13/001,632

(22) PCT Filed: Jun. 29, 2009
(86) PCT No.: PCT/IL2009/000646

§ 371 Date: Dec. 28, 2010

Related U.S. Application Data
(60) Provisional application No. 61/076,651, filed on Jun. 29, 2008.

(51) Int. Cl. G01N 33/53; C12M 1/34

(52) U.S. Cl. 435/7.21; 435/287.1

(57) ABSTRACT

One aspect of the disclosure is a method of characterizing a surface. In an exemplary embodiment, the method includes: (a) positioning a piezoelectric sensor near the surface; (b) obtaining by the sensor a resonant acoustic profile (RAP) of the surface; and (c) analyzing the obtained RAP to characterize the surface. The piezoelectric sensor has an interacting layer, which is adapted for binding to the surface to be characterized. In one embodiment, the characterized surface and the interacting layer are of about the same size.

### FIELD OF THE INVENTION

The present invention concerns material characterization with piezoelectric sensors, for example, quartz crystal microbalance (QCM). Particularly, but not exclusively, the invention concerns characterization of biological material.

### RELATED ART

A quartz crystal microbalance (QCM) measures a mass per unit area by measuring the change in frequency of a quartz crystal resonator. The resonance is disturbed by the addition or removal of a small mass due to film deposition at the surface of the acoustic resonator. The QCM can be used under vacuum, in gas phase and more recently in liquid environments. In liquid, it is highly effective at determining the affinity of molecules (proteins, in particular) to surfaces functionalized with recognition sites.

Frequency measurements are easily made to high precision; hence, it is easy to measure mass densities down to a level of below 1 μg/cm². In addition to measuring the frequency, the dissipation is often measured to help analysis. The energy of dissipation is a parameter dictated by the viscoelastic properties of the analyte and can assist in quantifying the damping in the system.

US Patent Application Publication No. 2004-0235198 describes a QCM biosensor for cancer diagnosis. The publication suggests cancer diagnosis based on acoustic resonance profiles of cells taken in biopsy, suspended, and seeded onto a chemically modified conducting surface of the QCM sensor.

### BRIEF SUMMARY

An aspect of some embodiments concerns a method of characterizing a surface. The method comprises:

positioning a piezoelectric sensor near the surface;

obtaining by the sensor a resonant acoustic profile (RAP) of the surface; and optionally analyzing the obtained RAP to characterize the surface.

In some embodiments, the piezoelectric sensor comprises an interacting layer adapted for binding to the surface. Optionally, the area of the interacting layer is about the same as the area of the characterized surface.

Optionally, the sensor is larger than the interacting layer, in a factor of 2, 5, 10, or any intermediate number.

Alternatively, the sensor is smaller than the interacting layer, in a factor of 2, 5, 10, or any intermediate number.

In some exemplary embodiments, the method comprising obtaining an RAP of the surface when the distance between the surface and the interacting layer is between 3 and 40 micrometers, for example, between 5 and 20 micrometers, such as 15 micrometer.

Optionally, the space between the sensor and the characterized surface is filled with aqueous solution, for example, OPTIMEM. This may be advantageous when the characterized surface comprises live cells or tissue.

Alternatively, the space is filled with gas, for example air. In some embodiments, the space is practically empty (vacuum).

In some preferred embodiments, the surface comprises a layer, optionally intact layer, of cells facing the piezoelectric sensor when said RAP is obtained.

Optionally, the cells are adhered to a rigid surface.

In some embodiments, the intact layer of cells is of a tissue, and the method is carried out in vivo or ex vivo.

Alternatively or additionally, the cells are of a diseased tissue.

In some embodiments, the interacting layer comprises synthetic molecules.

Additionally or alternatively, the interacting layer comprises molecules not obtained from a biological matter.

Additionally or alternatively, the interacting layer comprises a compound designed to interact with the analyte surface.

An aspect of some embodiments concerns a package containing a disposable portion of a piezoelectric sensor, also referred herein as a capsule. The disposable portion optionally comprises a piezoelectric element, and an electrode abutting said element and covered with an interacting layer.

Optionally, the package is sterile.

Optionally, the interacting layer is adapted for interacting with given marker molecules, and the package carries an indication to the identity of these marker molecules.

An aspect of some embodiments concerns a kit comprising a plurality of packages as described above.

Optionally, the kit comprises instructions for using the disposable portions to identify a specified tissue abnormality.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic illustration of a cross-section in a disposable portion of a piezoelectric sensor according to an exemplary embodiment;

FIG. 1B is a block diagram illustrating a system for identifying in vivo diseased tissue utilizing marker molecules to the diseased tissue, according to some embodiments of the invention;

FIG. 1C is a scheme showing three steps chemical immobilization of protein to gold surface: (1) thiol binding; (2) surface activation; and (3) protein coupling;

FIG. 2C is a schematic illustration of a sensing chamber for measuring of cells in suspension, by a sensor according to some embodiments;

FIG. 3A is a laser-confocal microscopy image of B16F10 cells labeled with a triplex composed of vitronectin, Rabbit monoclonal [EP873Y] anti Vitronectin and TxRed-conjugated anti rabbit IgG (the bar represents length of 20 μm);

FIG. 3B is a light epi-microscopy image (Magnification ×5) of B16F10 cells in suspension bound to the piezoelectric sensor;

FIG. 3C is a zoom in of FIG. 3B;

FIG. 4A is a graph showing the time-evolution of the frequency of a piezoelectric sensor having an interacting layer with vitronectin; curve (1): upon interaction with OPTIMEM (without cells); curve (2): upon interaction with 5×10⁵ B16F10 cells presuspended in OPTIMEM;

FIG. 4B is a graph showing the time-evolution of the resistance of a piezoelectric sensor having an interacting layer with vitronectin; curve (1): upon interaction with OPTIMEM (without cells); curve (2): upon interaction with 5×10⁵ B16F10 cells presuspended in OPTIMEM;

FIG. 5 is a schematic illustration of a sensing chamber for sampling and measuring an intact layer of cells adhered to a large rigid hydrophobic surface;

FIG. 6A is a graph showing the time-evolution of the frequency of a piezoelectric sensor upon interaction with 5×10⁵ B16F10 cells presuspended in OPTIMEM; curve 1: sensor without interacting layer; curve 2: sensor with vitronectin-containing interacting layer; and

FIG. 6B is a graph showing the time-evolution of the resistance of a piezoelectric sensor upon interaction with 5×10⁵ B16F10 cells presuspended in OPTIMEM; curve 1: sensor without interacting layer; curve 2: sensor with vitronectin-containing interacting layer;

### DETAILED DESCRIPTION

### Overview

An aspect of some embodiments of the invention concerns a method of piezoelectrically characterizing a subject material also referred herein as an analyte. In particular, but not exclusively, the subject material comprises tissue or cells adhered to a substrate. In some embodiments, the subject material comprises a substrate, optionally having a form of a flat surface. In one such example the subject material comprises cells adhered to a cover glass on which they grow.

Optionally, the piezoelectrical characterization utilizes a piezoelectric material connected to an electrode, such that when the electrode is electrified, the piezoelectric material changes its dimensions. Alternating voltage causes the piezoelectric material to vibrate. The vibration frequency of the piezoelectric material depends, inter alia, on the mass of the electrode attached thereto. Thus, when the electrode binds to compounds in its vicinity, the mass of the electrode changes, and with it the vibration frequency of the piezoelectric material.

It is generally preferred to use electrodes of inert electrically conductive materials, for example, gold, for the electrode.

Optionally, one side of the electrode is attached to the piezoelectric material and the other side—to a layer that specifically interacts with the subject material, referred to herein as interacting layer.

In some embodiments, the interacting layer comprises compounds that specifically bind to marker compounds appearing on the outer surface of cells. In some embodiments, the marker is specific to one kind of cells, and the results are analyzed to estimate the concentration of such cells in the subject material.

In some embodiments, the interacting layer comprises compounds that specifically interacts with marker compounds appearing on the outer surface of many kinds of cells, and the results are analyzed for estimating other characteristics of the subject material, for example, its viscoelasticity.

Optionally, the interacting layer is made of several portions, each with other interacting moieties. Optionally, this arrangement allows characterizing concurrently several characteristics of the subject material. Optionally each of the portions is continuous. Alternatively, at least one of the portions is non-continuous and has sub portions spaced apart from one another.

In some embodiments, the sensor is made of a disposable component and a non-disposable component. The disposable component, also referred herein as a capsule, comprises the piezoelectric element, the electrode, and the interacting layer.

The non-disposable component comprises the control unit, optionally including a power source and a processor for collecting and/or analyzing the results. Optionally, the capsules are provided packed in a packaging, optionally sterile packaging, and ready for use. In some embodiments, the package also contains instructions for using the capsule, for instance, instructions regarding the frequency to be applied to the capsule for identifying certain analytes, for characterizing a specified subject material, or the like.

Optionally, the package also contains an example of expected RAP. For example, the package may contain a profile typical for cancerous cells and a profile typical of non-cancerous ones.

For identifying cancer using profiles obtained in accordance with embodiments of the present invention, various characteristics may be used. For example, the acoustic profile obtained may be used to estimate the viscoelastic properties of the tissue or other biophysical properties, which distinguish malignant from benign cells.

In another example, the profiles may be used for testing the metabolic state of the tested cells or tissues. For instance, an interacting layer of metabolites that cancerous cells intend to excessively bind and insert, for example fluoro-deoxyglucose—FDG-18, is useful for identifying such cells with profiles obtained with some embodiments of the present invention.

In another example, the profiles may be used for testing the biochemical features of the cellular surface. For instance, certain cancerous cells are transformed in the expression of surface macromolecules set and exhibits cancer markers on their surface. An interacting layer designed to interact with such markers may be used for obtaining profiles indicative of the malignant state of the cells.

Since the electrode is preferably inert, in many embodiments the interacting layer is adhered to the electrode with an adhering layer. In case the electrode is made of gold, the adhering layer optionally comprises thiols.

To reduce the influence of a possibly noisy environment, in some embodiments the measurement is taken against a reference, exposed to similar noise. Optionally, the reference is a piezoelectric sensor identical to the measuring sensor, but without the interacting layer. Alternatively or additionally, the reference is a sensor contacting clear water or other reference material.

In some embodiments, the results of the measurement, optionally together with results of a reference measurement, are displayed as graphs showing the time evolution of the frequency and/or the resistance of the sensor following the contact of the sensor to the subject material.

The results of the measurements are optionally analyzed to estimate some characteristics of the subject material. Some conditions that the results may negate or confirm include cancer, psoriasis, Papilloma virus infected tissue, and other tissue infections and abnormalities.

An aspect of some embodiments of the invention concerns a sensing device for in vivo and/or ex vivo characterization of tissue, for example, for identification of diseased tissue, utilizing molecules appearing on the surface of cells of tissue to be characterized, followed by assessment of biophysical, biochemical, and/or metabolic features or state of the cells.

One embodiment provides a sensing device having a physical module and a biological module attached to the physical module, optionally by covalent bonding. The term biological module is used to denote that the module interacts, for example, binds to biological matter, and optionally is adapted to specifically bind to a given biological matter. An interactive layer as described herein is an example of a biological module.

The physical module comprises a sensor based on quartz crystal microbalance (QCM) or other piezoelectric sensor, which are collectively referred herein as QCM based sensors. The QCM based sensor is connected to a control unit comprising a resonant acoustic profiling unit.

The biological module comprises an interacting layer, having a plurality of nanstructures adapted to interact with the tissue (referred herein also as biomolecular structures). Optionally, the interacting layer comprises regions with high affinity to marker molecules in the tissue to be characterized. The high affinity regions specifically interact with the tissue.

Another aspect concerns a method of characterizing tissue, ex vivo and/or in vivo. The tissue characterized ex vivo is optionally, but not necessariloy, of dead cells.

In one embodiment, the method comprises attaching a piezoelectric sensor to the tissue, obtaining by the sensor a resonant acoustic profile of the tissue, and analyzing the profile to characterize the tissue.

Similarly, some embodiments provide a method of characterizing surfaces, having size comparable to the size of the sensor's electrode. Such surfaces are referred herein as large surfaces.

In an exemplary embodiment, such method comprises attaching a piezoelectric sensor to the large surface, obtaining, by the sensor, a resonant acoustic profile of the large surface, and analyzing the profile to characterize the large surface.

In the present specification, attaching a sensor to a surface, tissue, or any other subject material to be characterized, comprises bringing the sensor and the subject material to a distance between them, which is small enough to allow the sensor to sense the subject material. The specific distance optionally depends on the kind of subject material and on the specific sensor applied. Many times the subject material is rough on the scale of attachment distances. For example, in some embodiments, the distance between a cell carrying surface and an electrode is 15 micrometers, and the cells carried by the cell carrier are 15 micrometers in diameter. In such a case, the sensor and the cells actually touch each other, although the sample-sensor distance may be defined as 15 micrometers.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as necessarily limiting.

### Exemplary Piezoelectric Sensor

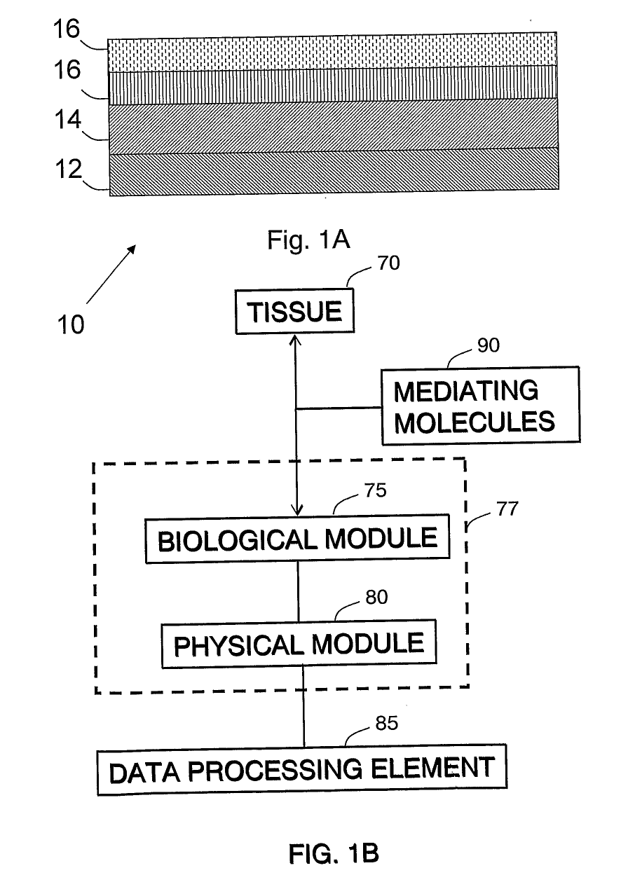

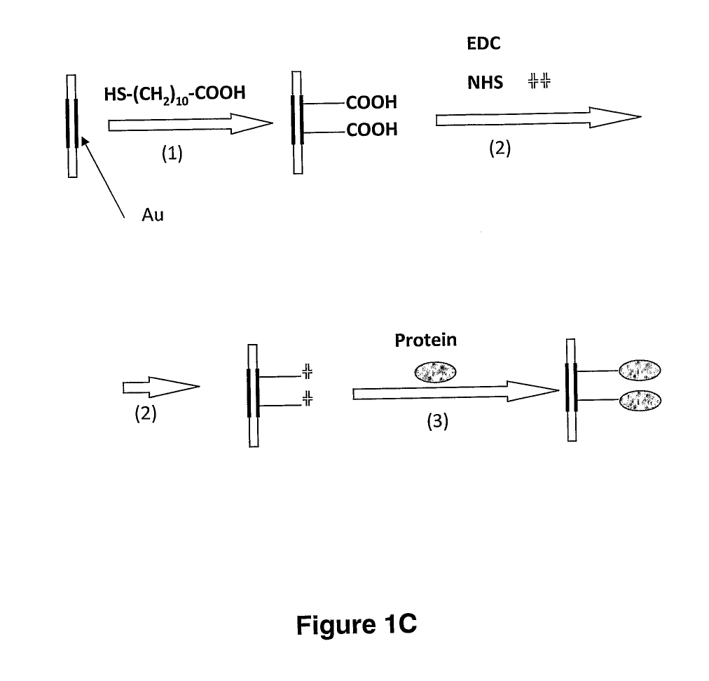

FIG. 1A schematically describes a piezoelectric sensor (10) suitable for use in a method according some embodiments. The figure shows a piezoelectric element (12) having one face abutting an electrode (14). Electrode 14 is connectible to a source of alternating electric field (not shown), and when the electrode is electrified, the piezoelectric element vibrates in a typical frequency, which depends on the mass of the element and the electrode attached thereto.

A face of electrode 14, opposite to the one abutting piezoelectric element 12 is functionalized with an interacting layer 16, designed to interact specifically with the surface (not shown) to be characterized by sensor 10.

Optionally, interacting layer 16 is adhered to the electrode with an adhering layer (18). The adhering layer is usually made of bifunctional molecules, having one functional group especially adapted for adhering to the electrode, and a second functional group adapted to adhere to components of the interacting layer. For example, in case electrode 14 is made of gold, adhering layer 18 optionally comprises compounds that have a functional group that adsorbs gold, such as thiols, and another functional group, for binding the interacting components of the interacting layer. In case the interacting layer comprises protein, the functional group is, for example, COOH or —NH₂.

### Examples of Piezoelectric Element

Optionally, piezoelectric element 12 comprises a quartz crystal, optionally of 0.2 mm thickness. Alternatively or additionally, the piezoelectric element 12 comprises other piezoelectric substances, some non-limiting examples of which include Zinc oxide, aluminum nitride, lithium tantalate polyvinylidene fluoride, aluminum orthophosphate, AlPO₄, gallium orthophosphate (GaPO₄), alumina borosilicate with fluorine, Tourmalines such as: Na(Al,Fe,Li,Mg,Mn)M₃Al(Si₆O₁₈)(BO₃)₃(OH,F)₄, XY₃Z₆[(BO₃)3Si₆O₁₈(OH, F)⁴] with X comprising K or Mn, Y comprising Mg, Al, Mn or Fe}^{II+}, and Z comprising Al, Mg, Ti, CR, V, Fe^{III+}. Further alumina borosilicate with fluorine, lanthanum gallium silicate, potassium sodium tartrate, or ceramics with perovskite tungsten-bronze structures, such as BaTiO₃, KNbO₃, Ba₂NaNb₅O₅, LiNbO₃, SrTiO₃, Pb(ZrTi)O₃, Pb₂KNb₅O₁₅, LiTaO₃, BiFeO₃, NaₓWO₃.

### Examples of Interacting Layer

In some embodiments, the interacting layer comprises compounds that specifically bind to marker compounds appearing on the outer surface of cells.

For example, for detecting cells with outer surface comprising receptors for a certain ligand, the interacting layer optionally comprises the ligand. More generally, the interacting layer optionally comprises substances that specifically interact with marker molecules appearing on the outer surface of cells.
Some examples of compounds that may be useful as ingredients in an interacting layer include:

Sugars for sensing metabolic state, characteristic of cancerous cells

Peptides with high affinity to cancer surface markers for sensing biochemical features of the membrane, characteristic of cancerous cells.

Peptides with high affinity to elements in Papilloma virus, for example, antibodies directed against Papilloma virus capsid oligemers, for biochemical sensing of Papilloma elements on the cell surface, characteristic of Papilloma virus infected cells.

Peptides directed to interact with generic cellular surface elements such as proteins, glycoproteins, phospholipids etc. for sensing biophysical features (such as viscoelastisity) of tissue, thereby characterizing the cell as malignant or benign.

Using interacting layers that are specific to certain kind or kinds of cells may be useful in characterizing whether the characterized sample contain such cells.

In some embodiments, the interacting layer comprises compounds that specifically interact with marker compounds appearing on the outer surface of many kinds of cells, and the results are used not to tell if any of these cells exist, but rather to estimate other characteristics of the subject material, for example, its viscoelasticity. Some examples of interacting materials suitable for the estimation of viscoelasticity of cells include antibodies directed against generic adhesion molecules, against the non-variable elements of MHC class I, ligands directed against generic receptors such as Vitronectin and Laminin, and/or any shorter functional derivate of these materials.

In some embodiments, the interacting layer comprises ingredients extracted obtained from biological material, for example, by extraction, purification, or the like. In some embodiments, the interacting layer comprises ingredients that were prepared synthetically from smaller base units, as generally known in the art of chemical synthesis.

In some embodiments, the interacting layer comprises ingredients that are not usually found in biological material, for example Polydipyrrole- and polydicarbazole-nanorods. Examples of non-biological materials designed to interact with biological materials may be found in CHEM COMMUN (CAMB). 2005 Sep. 14; (34):4357-9. Epub 2005 Jul. 14.

Optionally, the interacting layer is wholly or partially inorganic. Some inorganic materials suitable for interacting with biological materials are described, for instance, in US Patent Application Publication 20060047067 titled “Novel electroconductive polymers” to Jean Paul Lellouche.

### Exemplary References

To reduce the influence of a possibly noisy environment, in some embodiments the measurement is taken against a reference, exposed to similar noise. Optionally, the reference is a piezoelectric sensor identical to the measuring sensor, but without the interacting layer. Alternatively or additionally, the reference is a sensor contacting clear water or other reference material.

In some embodiments, the results of the measurement, optionally together with results of a reference measurement, are displayed as graphs showing the time evolution of the frequency and/or the resistance of the sensor following the contact of the sensor to the subject material.

### An Exemplary Device

The present invention discloses, inter alia, a sensing device and a method for identifying diseased tissue in vivo. The sensing device comprises a physical module, e.g. a quartz crystal microbalance sensor or other piezoelectric sensor connected (or at least connectible) to an integration and reporting box; and a biological module of biomolecular structures or other interacting layer connected to the physical module.

The biomolecular structures optionally comprise regions with high affinity to marker molecules for the diseased tissue. The sensor may be integrated in a disposable capsule and be fitted to identify different diseases.

FIG. 1B is a block diagram illustrating a system for identifying in vivo diseased tissue utilizing marker molecules to the diseased tissue, according to some embodiments of the invention. The sensing device 77 comprises a biological module 75 connected to a physical module 80 (e.g. covalently linked). Biological module 75 interacts with tissue 70 directly or in combination with mediating molecules 90, which optionally are markers residing at the tissue. Physical module 80 is connected to a data processing element controlling and analyzing the measurements.

According to some embodiments of the invention, physical module 80 may comprise a quartz crystal sensing device characterized by high specificity and sensitivity (e.g. the order of magnitude of 1 ng/cm²). Biological module 75 may comprise peptides or peptide like structures (like antibodies or ligands), that comprise at least one high affinity region to surface expressed markers of diseased (e.g. malignant) tissue.

According to some embodiments of the invention, mediating molecules 90 may comprise molecules to which diseased cells or tissue have an aberrant affinity, such as sugar (for cells with an elevated metabolic rate, e.g. FDG—Fluorodeoxyglucose), ligands (for receptors that are over-expressed in certain diseased cells, such as ErbB₂ in breast cancer.

According to some embodiments, mediating molecules 90 may comprise marker molecules to specific diseases.

According to some embodiments, mediating molecules 90 may comprise molecules binding to all cells (e.g. to proteins encoded by the MHC I—major histocompatibility complex class I) and allowing an indication of cell rigidity.

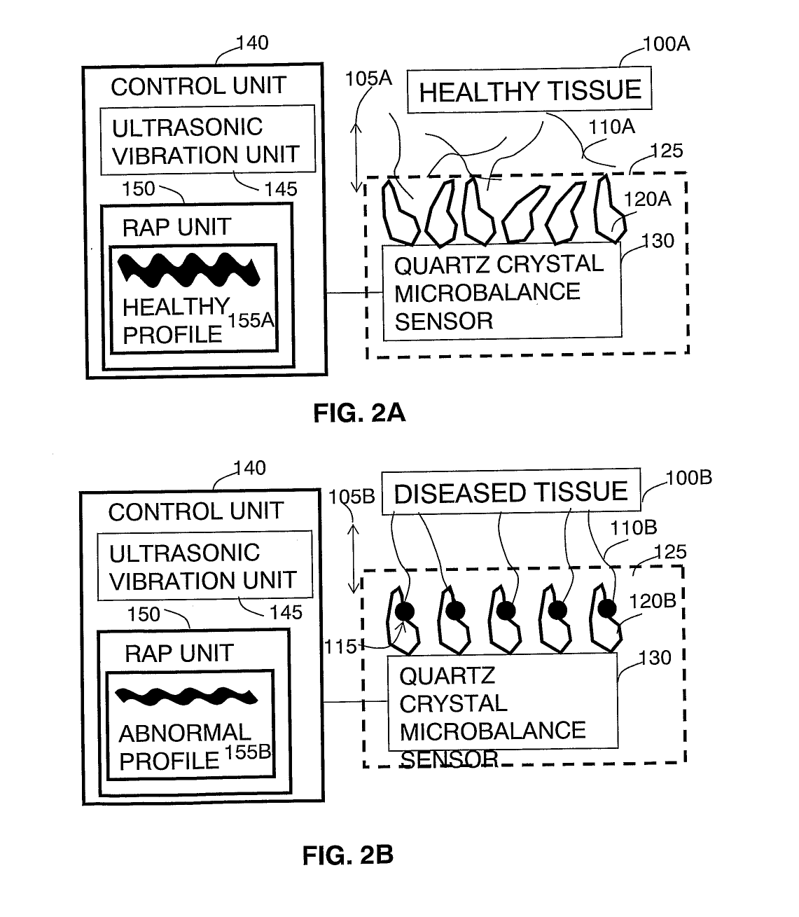

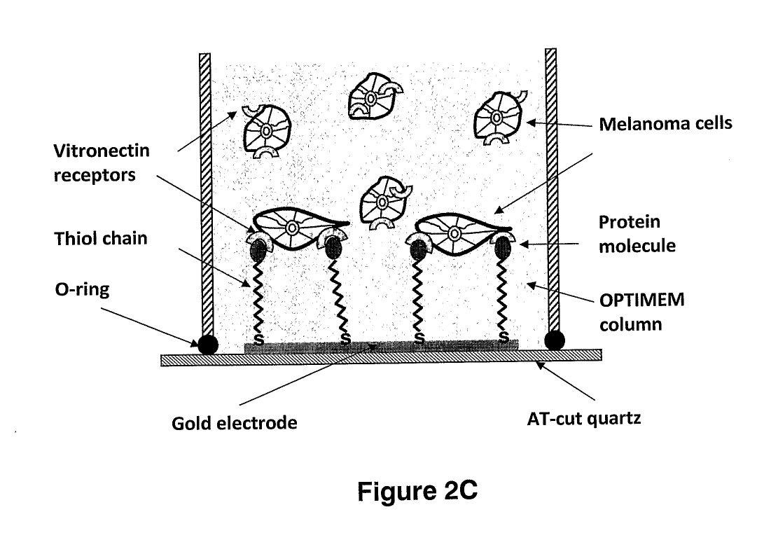

FIGS. 2A and 2B are schematic illustration of a sensing device 125 for identifying in vivo diseased tissue 100B utilizing marker molecules to diseased tissue 100B, according to some embodiments of the invention. FIG. 2A illustrates sensing device 125 not binding to a healthy tissue 100A, while FIG. 2B illustrates sensing device 125 binding to a diseased tissue 100B. The configuration of elements in relation to healthy tissue 100A are marked with an “A” (e.g. “110A”, “120A”). The configuration of elements in relation to diseased tissue 100B are marked with a “B” (e.g. “110B”, “120B”). The sensing device 125 comprises a physical module comprising sensor based on quartz crystal microbalance 130 and a biological module comprising a plurality of biomolecular structures 120, making together an interacting layer, which are connected to quartz crystal microbalance sensor 130. Quartz crystal microbalance sensor 130 is connected to a control unit 140. Control unit 140 comprises an ultrasonic vibration unit 145 and an RAP (resonant acoustic profiling) unit 150 with a measurement profile display 155. Biomolecular structures 120 may interact with tissue 100 directly (not shown) or in combination with mediating molecules, e.g. marker molecules 110. Biomolecular structures 120 comprise regions 115 with a high binding affinity to marker molecules 110 for the diseased tissue 100B. When healthy tissue 100A is measured by sensing device 125, no complex is formed between tissue 100A, marker molecules 110A and biomolecular structures 120A. Measurement profile display 155A displays a healthy profile. When diseased tissue 100B is measured by sensing device 125, marker molecules 110B bind to diseased tissue 100B and to biomolecular structures 120B, and form a complex. Measurement profile display 155B is of an abnormal profile. The complex changes the frequency and frequencies span measured by quartz crystal microbalance sensor 130 and the measurements are analyzed by control unit 140 to diagnose diseased tissue 100B.

The results of the measurements are optionally analyzed to estimate some characteristics of the subject material. Some conditions that the results may negate or confirm include cancer, psoriasis, Papilloma virus infected tissue and other tissue abnormalities.

According to some embodiments of the invention, inspection and diagnosis of suspected tissues for malignancy is carried out in on-line, by monitoring the intact tissue without the need for any surgical procedure, or any biological, chemical, irradiative or radioactive labeling, or scanning.

In some embodiments, characterizing a tissue includes identifying if a tissue has a certain disease. In an exemplary embodiment, there is provided a method of confirming (or negating) a diseased state of a tissue, which method comprises
defining the type of disease to be detected;
choosing marker molecules and a sensor capsule relating to the type of disease to be detected;
attaching the sensor capsule to a control unit comprising a resonant acoustic profiling unit;
attaching the sensor capsule to the tissue in vivo in a way that the biomolecular structures of the interacting layer interact with the marker molecules on the tissue to a resonant acoustic profile;
analyzing the resonant acoustic profile by the resonant acoustic profiling unit; and optionally
disposing the capsule.

### EXAMPLE

Reference is now made to the following examples, which together with the above descriptions illustrate some embodiments of the invention in a non limiting fashion.

In the following example an intact live layer of melanoma cells adhered to a large surface is sampled by a piezoelectric sensor, in a measurement sensitive to the biochemical properties and viscoelasticity of the cells.

### Materials.

Thiol (HS(CH₂)₁₀CO₂H) 11-Mercaptoundecanoic acid 95%, N-(3-Dimethylaminopropyl)-N′-ethylcarbodiimide hydrochloride, N-Hydroxysuccinimide, bovine serum albumin (BSA), paraformaldehyde (PFA), were purchased from Sigma-Aldrich. PBS buffer purchased from Biological-Industries (Beit-Haemek, Israel). OPTIMEM manufactured by Rhenium (Sigma-Aldrich); Lipohilized vitronectin manufactured by Biopur (BL Switzerland). 13 mm diameter cover glasses manufactured by Marienfeld (Germany). All the other chemicals that were used are analytical grade from local suppliers.

Antibodies. Primary antibody (1⁰)—Rabbit monoclonal [EP873Y] anti Vitronectin by Abcam (Cambridge UK). Secondary Antibody (2⁰)—Goat anti rabbit conjugated Texas red manufactured by Enco materials (Jackson Tenn.).

### Cell Cultures and Growth Conditions

B16F10 cell-line supplied by ATCC (VA USA), derived from mice's melanoma, were cultured in the standard conditions in stock dishes (10 cm²) in a humidified incubator with 5% CO₂/95% air atmosphere at 37° C. Cells were grown in DMEM 4.5 gr/L glucose medium (Biological-Industries Beit-Haemek, Israel). This culture medium was additionally supplemented with 0.6 g/l glutamine, 10% (v/v) fetal bovine serum, 0.1 g/l pyruvate, 100 unit/l penicillin, 0.1 g/l streptomycin and 4 mg/l gentamycin. All experiments were conducted on the 5^{th} passage.

### Indirect Immunofluorescence

Cells were seeded on pre-cleaned cover glasses at concentration 1×10⁵/0.5 ml/well and incubated at 37° C. overnight. Then, the cells were washed with OPTIMEM and starved in OPTIMEM, for 30 minutes. Then, the cells were incubated for 2 h in 37° C. with 0.2 μg/cm² Vitronectin in OPTIMEM. Then, the cells were washed with PBS and fixed in fixation buffer; PFA (4%) Glutaraldehyde (0.1%) Sucrose (4%) dissolved in PBS. Cells were subsequently incubated overnight at 4° C. with 1° antibody directed against vitronectin, diluted in 0.1% BSA, 4% NGS/PBS, followed with 1 h incubation with the appropriate secondary antibody conjugated to Texas Red. Cover-glasses were subsequently washed and mounted with Gel Mount mounting medium (Biomeda, Foster City, Calif.). Samples were analyzed using an Olympus laser confocal microscope.

### Sensor System Setup

The SRS QCM200 sensor system (Stanford Research Systems, Sunnyvale USA) used herein is a stand-alone instrument with a built-in frequency counter and resistance meter. It includes controller, crystal oscillator electronics and crystal holder. The sensor data is transferred from the frequency counter to laptop PC through RS232C serial ports and collected in real time by a computer module installed on the PC. The sampling period and the resolution of the frequency counter were 1 sec. and 0.1 Hz, respectively. All the measurements were done in incubator at 37° C. The oscillator units and 5-MHz QCMs with AT-cut quartz are from Stanford Research Systems, Inc. The electrode (active sensing area) of the QCM is 0.4 cm² in size. To exclude external influences and noises in the measurements, each experiment conducted simultaneously using two sensors in two separate crystal holders which were connected to one PC processing unit. One of each was used as a reference and the other one, as the experiments. As detailed below, the reference cell included in one case a sensor without the interacting layer, contacting cells; and in another case, a sensor with interacting layer, contacting a sample with no cells. Signals from both sensors were received and processed in real-time, by a program module, allowing on-line data reading.

### Vitronectin Immobilization

Protein was attached to the QCM electrode's gold surface using self-assembled monolayer method, based on the ability of alkane-thiol chain to adsorb onto a gold surface on the thiol end and bind to amine groups of proteins on the other end (see FIG. 1C). Crystals were cleaned with Piranha solution (30% H₂O₂ and conc. H₂SO₄ mixture in 1:3 by volume), washed and dried with Argon flow. 0.22 mg of 11-Mercaptoundecanoic acid was dissolved in 100% ethanol to create a 1 mM solution and the solution was applied to the crystals (mounted in the holders) for 2 hours at RT=24° C., crystals then were washed with ethanol and DDW (step 1). An activating mixture [8 mg of 1-Ethyl-3-(3-Dimethylamino-propyl) carbodiimide (EDC) and 13.5 mg of NHS(N-hydrocylsulfo-succinimide) dissolved in 0.4 ml of PBS] was used to activate the carboxyl groups of 11-Mercaptoundecanoic acid. The activating mixture was applied to the gold surface for 1 hour (step 2 in FIG. 1), subsequently, 5 μl of vitronectin stock solution (0.1 mg/ml in DDW) were added to the mixture for further 1 hour incubation. Then, the crystals were washed with PBS followed by final washing with OPTIMEM (step 3 in FIG. 1) and herein ready for measurements.

### Preparation of B16F10 Cells for QCM Measuring of Vitronectin—Cells Interactions

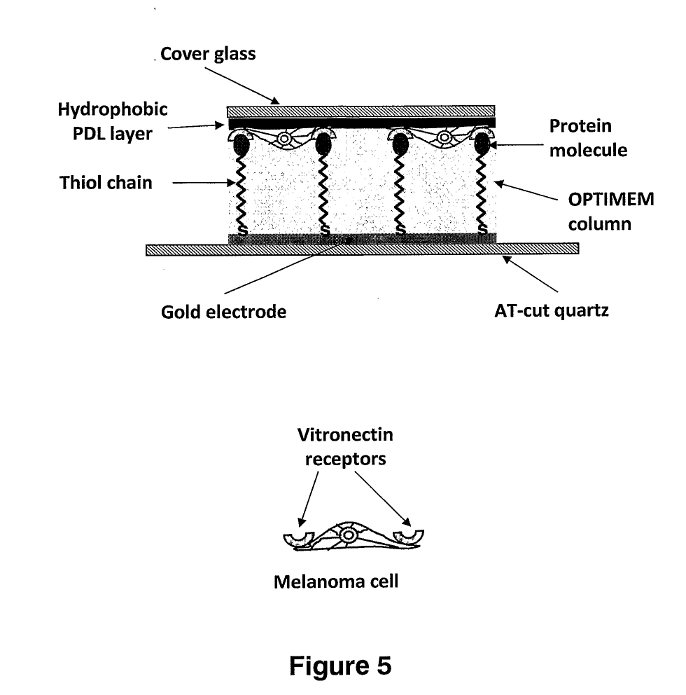

For measurements in suspension, B16F10 cells were detached from 10 mm plastic dishes (80% confluent) by washing with warmed PBS, followed by incubation in 2 ml of warmed PBS 0.05% EDTA for 5-10 min in 37° C. The obtained detachment solution was blocked with 8 ml of DMEM, 0.1% in BSA. Then, the cells were spinned down at 1500 rpm, for 5 min and re-suspended in warmed OPTIMEM to final concentration of 5×10⁵ cells/100 μl. 100 μl of the final suspension was submitted to the piezoelectric composed crystal (see FIG. 2). For the external monolayer QCM's examinations, cells 1×10⁵ were seeded on Ø13 mm cover-glass, pre-coated with poly-D-lysin (see FIG. 5).

### Results

### Vitronectin Binds the Focal Adhesion of B16F10 Cells

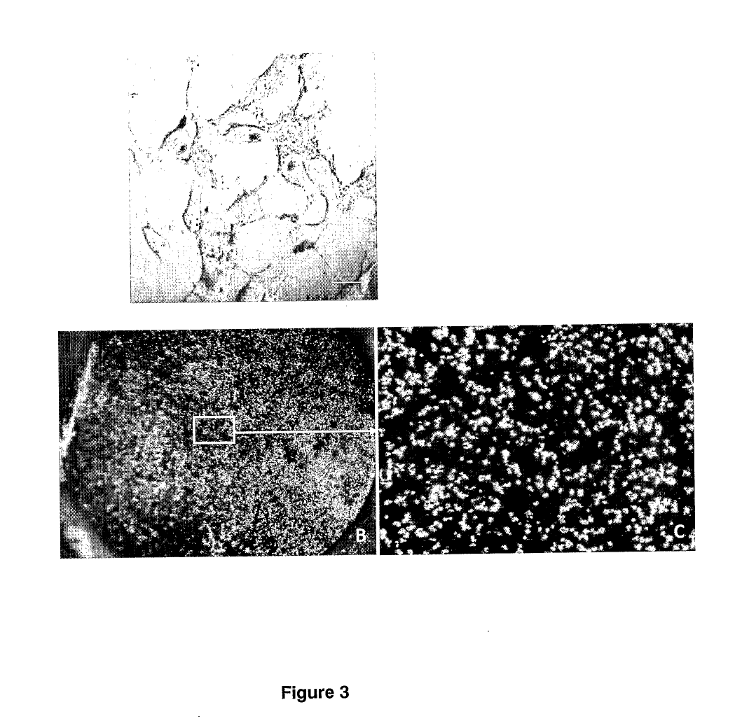

To investigate the localization and robustness of the vitronectin binding sites on B16F10 cells; vitronectin ligand was submitted to the cells. The bound vitronectin was labeld by indirect immunofluorescence. B16F10 cells were visualized by confocal microscopy, demonstrating a robust peripheral staining and staining at focal adhesion (see FIG. 3A).

From FIG. 3A one can see that the cell nuclei protrude out of the plane defined by the cells, and this might be an obstacle to good contact between the cell layer and the sensor surface. To cope with this difficulty, long thiol chains were used, deposited in large enough a distance from each other to allow the molecules to bend as necessary to attach to the protruding portions of the cells, or to stand upright as may be required for attaching to other regions of the cell layer.

The Sensor can Bind B16F10 Cells from Suspension

To examine the competence of our sensor to affix B16F10 cells, the electrode was coated with vitronectin through alkane-thiol chain (see vitronectin immobilization procedure). Then, suspension of cells was submitted to the sensor. Unbound cells were removed by washing, which did not remove any of and the bound cells. Visualizing the obtained electrodes in light epi-microscopy clearly demonstrated agglutination of cell clusters to the crystal surface (see FIG. 3B, C).

Cells Attached to the Sensor from Suspension Decreased the Observed Frequency and Elevated the Resistance

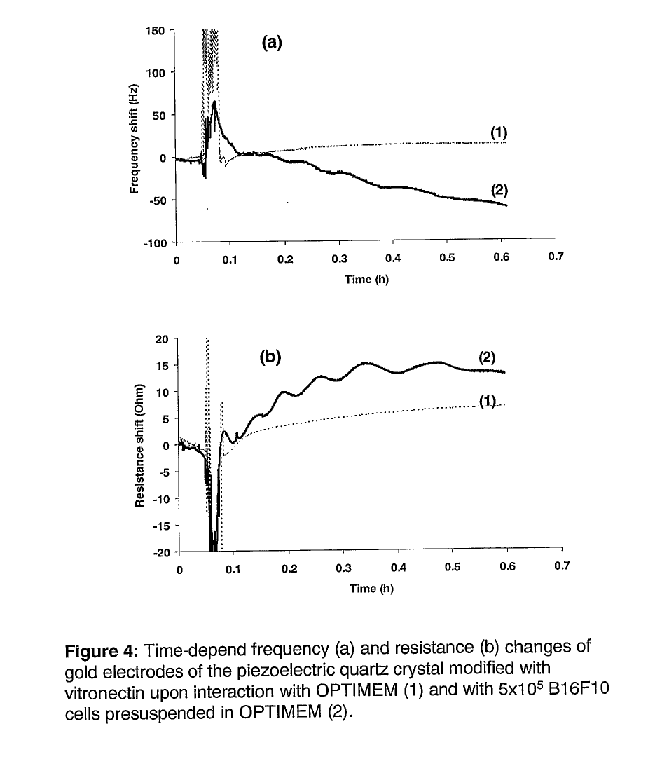

As visualized in FIGS. 3B and 3C, affixing B16F10 cells to a sensor having an electrode coated with vitronectin leads to changes in the piezoelectric properties of the sensor (see FIG. 4).

FIG. 4 demonstrates the response of a piezoelectric sensor to B16F10 cells in suspension (as illustrated in FIG. 2).

Curve 1 of FIG. 4 was produced by a reference sensor, to which a cell-free medium was added at a time that can be clearly identified on the graph due to sharp jumps in the frequency and resistance.

Curve 2 was produced by a sensor, to which a cell-containing medium was added at the same time. The time of adding the medium is clearly identifiable in curve 2.

The curves 1 and 2 are results of experiments that were carried out simultaneously under the same external conditions.

After medium addition, curve 1 shows a recovery of the system, which returns to its baseline values, of before the medium addition.

In curve 2, after medium addition, the frequency (FIG. 4A) drops and the resistance (FIG. 4B) increases, and both show sinusoidal fluctuations that may be explained by cell motion and different metabolic activities of cells near the sensing surface. The general increase in resistance (of about 13 Ohm, see line 2 of FIG. 4B) can perhaps be explained by the increased surface viscosity in the presence of cells.

An Intact Monolayer of B16F10 Cells on a Large Rigid Surface can be Placed on a Piezoelectric Sensor Allowing Binding of Cells to the Large Rigid Surface on One End and to the Sensor on the Other

The competence of the piezoelectric sensor to acoustically profile a monolayer of living cells, forming an intact monolayer on a large rigid surface, required a preliminary examination to verify that a live intact monolayer neither blocks the instrument, nor prevents attachment of cells to the sensor. For these purposes, B16F10 were seeded, and raised to form confluent monolayer on Ø13 mm cover-glass precoated with poly-D-lysin. The cells monolayer was then placed on a piezoelectric sensor with a determined volume of medium (OPTIMEM) (see FIG. 5). The entire volume of the medium had remained located in between the two surfaces (sensor and cover-glass) due to the capillary effect, which prevented OPTIMEM from smearing away. Moreover, the hydrophobic quality of the poly-D-lysin layer had amplified this capillary effect, by increasing surface tension. Hence, determination medium's volume allowed good estimation of the hight of the medium column between the sensor and the large rigid surface to which the cells were attached. For example, the cover glass diameter was 13 mm=1.3 cm, corresponding to a surface area of A=πr²=1.33 cm². For instance, providing 5 μl=5×10⁻³ cm³ of medium on the sensor and covering it with the cover-glass, results in liquid column having a height of 5×10⁻³ cm³/1.33 cm²=37.6 μm, which is the distance between the sensor and the cover-glass (see FIG. 5). In practice, after the medium was provided, small portions thereof were removed with gentel peristaltic pumping, to bring the surfaces closer together. After each removal of about 0.5 μl medium, the system was left for equilibration during several minutes, and if no frequency change was observed, further medium was removed. If the slightest change in frequency was observed and remained stable for a few minutes, no more medium was removed, and the remaining of the measurements were carried out at constant distance between the cell carrying surface and the sensor.

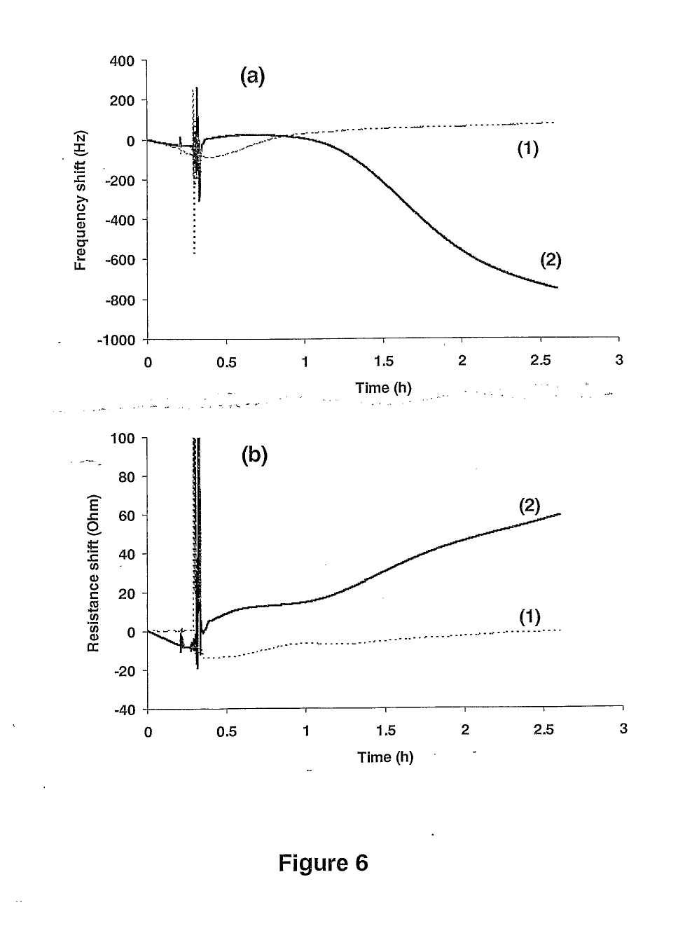

Curves 1 in FIGS. 6A and 6B reveal that contacting cells with a system similar to that schematically described in FIG. 5, but without the interacting layer, does not change the reading of the sensor, other than the initial perturbation caused by medium introduction. Curves 2 in FIGS. 6A and 6B show that contacting cells with a system similar to that schematically described in FIG. 5, including the interacting layer, does change the reading of the sensor, and causes a frequency decrease and resistance increase. These phenomena can be explained by the binding of vitronectin to the cells through vitonectin's binding sites.

FIG. 5 is a schematic illustration of the system obtained during the experiments, the results of which are depicted in FIG. 6.

The figure shows a gold electrode (502) abutting an AT-cut quartz crystal (504), used as a piezoelectric element. Electrode 502 has on it a proteinic layer of vitronectin molecules (506) attached to the electrode by thiol molecules (508). On piezoelectric element 504, rests a column of OTIMEM (510), held in place by capillary forces between the electrode and a layer of cells (512), simulating a tissue. Cells 512 are attached to a cover glass (514) through a hydrophobic layer 516 and with their vitronectin receptors (518) to thiols 508.

A Monolayer of B16F10 Cells Attached to a Large Rigid Hydrophobic Surface Changes the Piezoelectric Properties of the Sensor

The results disclosed herein show that the inventors succeeded in finding conditions, under which piezoelectric sensor can measure intact live layer of cells adhered to a surface external to the sensor.

In more detail, lines 2 of FIGS. 6A and 6B present measurement results obtained by a QCM sensor having a vitronectin-containing interacting layer sensing an intact layer of B16F10 cells adhered to a large rigid surface. FIG. 6A exhibit an initial 10 min small decrease in frequency followed by a 45 min exponential decrease of frequency, which reaches about 760 Hz below the baseline, defined as the reading before the cells were introduced to the sensor.

FIG. 6B line shows a corresponding increase of resistance, which reaches up to about 45 Ohm above the baseline. After the exponential change, the measured frequency and resistance stabilized at new equilibrium values.

Furthermore, unpresented data show that the frequency and resistance fluctuates similarly to the fluctuations shown in FIG. 4.

One model that explains this behavior is that at first only some cell elongations reached out far enough to bind to the sensor. After some such bounds were made, it was easier for the entire cell as well as for adjacent cells to reach out, and thus increase the number of bonds between the sensor and the cell layer. Such mechanism would be expected to exhibit exponential behavior until a steady state is achieved. A similar, but less pronounce, behavior was found in QCM measurements of cells in suspension.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

To the extent that section headings are used, they should not be construed as necessarily limiting.

In the above description, an embodiment is an example or implementation of the inventions. The various appearances of “one embodiment,” “an embodiment” or “some embodiments” do not necessarily all refer to the same embodiments.

Although various features of the invention may be described in the context of a single embodiment, the features may also be provided separately or in any suitable combination.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

The term “exemplary” is used in the sense of serving as an example, and not necessarily as outstanding.

It is to be understood that the terms “including”, “comprising”, and grammatical variants thereof mean including, but not limited to, and do not preclude the addition of one or more components, features, steps, or integers or groups thereof and that the terms are to be construed as specifying components, features, steps or integers.

If the specification or claims refer to “an additional” element, that does not preclude there being more than one of the additional element.

It is to be understood that where the claims or specification refer to “a” or “an” element, such reference is not be construed that there is only one of that element.

It is to be understood that where the specification states that a component, feature, structure, or characteristic “may”, “might”, “can” or “could” be included, that particular component, feature, structure, or characteristic is not required to be included.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

1-11. (canceled)

12. A method of characterizing a surface, the method comprising:
positioning a piezoelectric sensor near the surface;
obtaining by the sensor a resonant acoustic profile (RAP) of the surface; and
analyzing the obtained RAP to characterize the surface,
wherein said piezoelectric sensor comprises an interacting layer adapted to bind to the surface and having an area that is not larger than 10 times the area of the surface.

13. A method according to claim 12, comprising obtaining an RAP of the surface when the distance between the surface and the interacting layer is between 3 and 40 micrometers.

14. A method according to claim 12, wherein said surface comprises a layer of cells facing the piezoelectric sensor when said RAP is obtained.

15. A method according to claim 14, wherein said cells are adhered to a rigid surface.

16. A method according to claim 14, wherein said cells are of a diseased tissue.

17. A sterile package containing a disposable portion of a piezoelectric sensor, said portion comprising a piezoelectric element, an electrode abutting said element and covered with an interacting layer.

18. A sterile package according to claim 17, wherein the interacting layer is adapted for interacting with given marker molecules, and the package carries an indication to the identity of said marker molecules.

19. A kit comprising a plurality of sterile packages, each according to claim 18.

20. A kit according to claim 19, comprising instructions for using the disposable portions to identify a specified tissue abnormality.
